(19) United States
(12) Patent Application Publication
(10) Pub. No.: US 2020/0222619 A1
(43) Pub. Date: Jul. 16, 2020

(54) Millimeter Wave Radiation of Blood Container

(71) Applicants: Iosif Mikhail Gershteyn, Waltham, MA (US); Mikhail Lev Gershteyn, Newton, MA (US)

(72) Inventors: Iosif Mikhail Gershteyn, Waltham, MA (US); Mikhail Lev Gershteyn, Newton, MA (US)

(21) Appl. No.: 16/246,473

(22) Filed: Jan. 12, 2019

(51) Int. Cl. A61M 1/36; A61M 1/14; A61M 1/38; A61M 39/08

(52) U.S. Cl. CPC A61M 1/3681; A61M 1/14; A61M 1/38; A61M 39/08; A61M 2205/04; A61M 2205/3375; A61M 2205/3306; A61M 2205/18; A61M 2209/088; A61M 2230/20

(57) ABSTRACT

Apparatuses and methods for millimeter or sub-millimeter wave radiation of blood outside the body. Electromagnetic (EM) radiation length may be preset by manufacturer, programmable by the user, or dependent on sensor reading(s) of blood parameters (viscosity, color, opaqueness). Quality control measures may include sensor blood readings prior to irradiation and after irradiation by electromagnetic waves. In one embodiment emitter is connected to catheter. Applications considered include but not limited to dialysis, blood transfusion. Radiation parameters (frequency, intensity, pulse duration) may be dependent on sensor readings of blood parameters (viscosity, color, and opaqueness) enabling fine-tuning of electromagnetic signal for maximal normalization of blood parameters (including viscosity and coagulation) specific to patient. Additional apparatuses considered where emitters radiate on the body directly, for applications including but not limited to decreasing edema and/or reducing pain, and/or reducing stiffness, and/or increasing blood circulation in targeted parts of the body (e.g. legs, arms) as needed.

### BACKGROUND

Millimeter wave electro-magnetic low-intensity radiation therapy has been used in a wide breadth of therapeutic applications including cardiovascular disease, diabetes, gastrointestinal disorder, and pain relief. Millimeter wave radiation of blood that is external to a human body may be useful.

### BRIEF DESCRIPTION OF THE DRAWINGS

Some embodiments of the technology are illustrated, by way of example and not limitation, in the figures of the accompanying drawings.

FIG. 1 illustrates an example system in which millimeter wave radiation of a blood container may be implemented, in accordance with some embodiments.

FIG. 2 illustrates an example system in which millimeter wave radiation may be applied to packed red blood cells, in accordance with some embodiments.

### SUMMARY

The present disclosure generally relates to millimeter wave radiation of a blood container.

As set forth above, millimeter wave radiation of blood that is external to a human body may be useful, for example, for improving blood's rheological properties including normalizing red blood cell aggregation rate. According to some aspects, an apparatus comprises: a container storing blood; means for magnetically attaching a device to the container; and means for emitting electromagnetic millimeter wave radiation on the container for a predetermined period of time.

According to some aspects, a system comprises: a container storing blood in an internal part of the container; and a device magnetically attached to the container at an external part of the container, the device emitting electromagnetic millimeter wave radiation on the container for a predetermined period of time.

According to some aspects, a system comprises: a container storing packed red blood cells in an internal part of the container; a first magnet attached to the container at a first external part of the container; a second magnet attached to the container at a second external part of the container; and a millimeter wave emitter coupled to the second magnet, the millimeter wave emitter emitting electromagnetic millimeter wave radiation on the container for a predetermined period of time.

### DETAILED DESCRIPTION

The present disclosure describes, among other things, techniques for millimeter wave radiation of a blood container. In the following description, for purposes of explanation, numerous specific details are set forth in order to provide a thorough understanding of the various aspects of different embodiments of the present disclosure. It will be evident, however, to one skilled in the art, that the present disclosure may be practiced without all of the specific details.

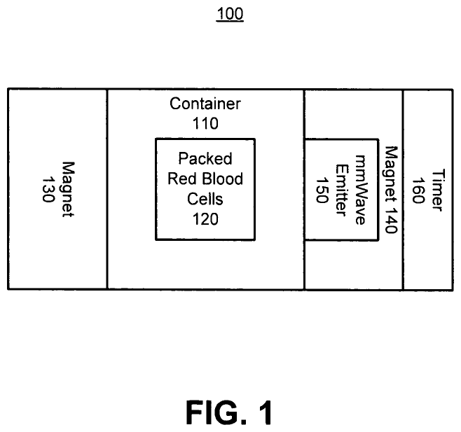

FIG. 1 illustrates an example system 100 in which millimeter wave radiation of a blood container may be implemented, in accordance with some embodiments. As shown, the system 100 includes a container 110, packed red blood cells 120, a magnet 130, a magnet 140, a millimeter wave emitter 150, and a timer 160.

The container 110 stores the packed red blood cells 120. In some examples, the packed red blood cells 120 may be replaced with blood plasma or any other blood. The container 110 may be made of plastic, glass, or any other material. An example blood container, which can serve as the container 110, is described in U.S. Pat. No. 3,217,710, issued to Beall on Nov. 16, 1965, which is incorporated herein by reference in its entirety. A first magnet 130 and a second magnet 140 are placed at the sides of the container, for example, at opposite sides. Any magnetic material may serve as the first magnet 130 or the second magnet 140. The container 110 may be a plastic or glass container which is external to and distinct from a human (or other animal) body.

The magnet 130 or the magnet 140 may be a material or object that produces a magnetic field. This magnetic field is invisible but is responsible for the most notable property of a magnet: a force that pulls on other ferromagnetic materials, such as iron, and attracts or repels other magnets.

As shown, the magnet 140 includes a millimeter wave (mmWave) emitter 140. The mmWave emitter 140 may include all or a part of a mmWave radio. Example mmWave radio(s) are described in U.S. Pat. No. 9,659,904, issued to Kamgaing on May 23, 2017, which is incorporated herein by reference.

The mmWave emitter 150 is coupled with a timer 160. The timer 160 is used to set a predetermined (e.g. by a user) period of time. The mmWave emitter 150 emits mmWave radiation on the container 110 for the predetermined period of time. The predetermined period of time may be programmed by a user.

In some aspects, the system 100 includes the container 110 storing blood (e.g. packed red blood cells 120) in an internal part of the container 110. The system 100 includes a device (e.g. mmWave emitter 150) magnetically (e.g. via magnet 140) attached to the container 110 at an external part of the container 110. the device emits electromagnetic millimeter wave radiation on the container 110 for a predetermined period of time (e.g. set by the timer 160).

In some aspects, the system 100 includes the container 110 storing packed red blood cells 120 in an internal part of the container 110. The system 100 includes the first magnet 130 attached to the container 110 at a first external part of the container 110. The system 100 includes the second magnet 140 attached to the container 110 at a second external part of the container 110. The system 100 includes a millimeter wave emitter 150 coupled (e.g. attached) to the second magnet 140. The millimeter wave emitter 150 emits electromagnetic millimeter wave radiation on the container 110 for a predetermined period of time. The predetermined period of time may be programmed by the user using the timer 160.

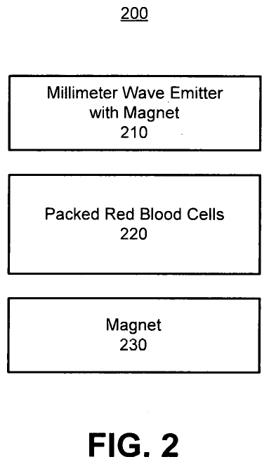

FIG. 2 illustrates an example system 200 in which millimeter wave radiation may be applied to packed red blood cells, in accordance with some embodiments. As shown, the system 200 include a millimeter wave emitter with magnet 210, packed red blood cells 220, and a magnet 230. The millimeter wave emitter with magnet 210 may correspond to the magnet 140 and mmWave emitter 150 of FIG. 1. The packed red blood cells 220 may correspond to the packed red blood cells 120 of FIG. 1. The magnet 230 may correspond to the magnet 130 of FIG. 1.

In some examples, the technology described herein relates to medicine and blood transfusion. Millimeter wave electro-magnetic low-intensity radiation therapy is useful in a wide breadth of therapeutic applications including cardiovascular disease, diabetes, gastrointestinal disorder, and pain relief. Millimeter wave radiation may be applied to the skin at such a low intensity that the patient does not feel any heating effect. Given that millimeter wave exposure intensity is <10 mW/square centimeter of skin, in accordance to United States Department of Labor Occupational Safety and Health Administration (OSHA) Regulations Standard 29 CFR 1910.97 there may be no harm to a person's health as this is non-ionizing radiation.

In some aspect, the technology described herein includes a method and device that improves blood's rheological parameter(s) prior to transfusion. Rheological parameter(s) effected by millimeter radiation of the blood in stored in pouch or other container (outside human organism), includes normalizing the rate of erythrocyte (red blood cell) aggregation thus increasing the exposed surface area of red blood cells. This enables the red blood cells to deliver more oxygen to tissue and may improve the clinical effect of blood transfusion for patients. In some cases, low-intensity millimeter-waves applied to blood cells in test tubes (“in-vitro”) improves rheological properties. In some cases, millimeter wave radiation is applied to the entire person (at the same frequency as “in vitro”), from patients who had stomach and/or duodenum ulcers.

One embodiment includes a magnetically attached device that would hug a pouch or container for red blood cells or plasma (for example standard packed red blood cells). Then the electro-magnetic emitter would be turned on for a programmable period of time (e.g. 5 minutes, 10 minutes, 15 minutes, etc.). This embodiment is shown in FIG. 2, and described above.

One type of transfusion is of Red Blood Cells (RBC). However, there is evidence that RBCs stored for over 2 weeks are damaged and do not improve the clinical outcome. In the United States, the Food and Drug Administration (FDA) mandates that the maximal allowable shelf life of stored human red blood cells (RBCs) requires maintaining cellular integrity (assessed as free hemoglobin <1% of total hemoglobin) together with an average 24-hour post-transfusion RBC survival of more than 75%. RBC survival may increase after exposure to low-intensity millimeter wave radiation because cell aggregation rate has been shown to be normalized ‘in vitro.’ Therefore RBC cells should be less aggregated, and have more surface area available for oxygen transport once they enter the organism, making them more efficacious for the patient. Increased RBC aggregation may be implicated in Gaucher disease, and sickle cell disease.

Erythrocyte aggregation is the reversible clumping of red blood cells (RBCs) under low shear forces or at stasis. Erythrocytes aggregate in a special way, forming rouleaux. Rouleaux are stacks of erythrocytes which form because of the unique discoid shape of the cells in vertebrate body. The flat surface of the discoid RBCs gives them a large surface area to make contact and stick to each other; thus, forming a rouleau. Rouleaux formation takes place in suspensions of RBC containing high-molecular, fibrilar proteins or polymers in the suspending medium. The most important protein causing rouleaux formation in plasma is fibrinogen. RBC suspended in simple salt solutions may not form rouleaux.

Conditions which cause increased rouleaux formation include infections, inflammatory and connective tissue disorders, and cancers. It also occurs in diabetes mellitus and is one of the causative factors for microvascular occlusion in diabetic retinopathy.

Erythrocyte aggregation is the main determinant of blood viscosity at low shear rate. Rouleaux formation also determines Erythrocyte sedimentation rate which is a non-specific indicator of the presence of disease.

Increased erythrocyte (red blood cell) aggregation rate may be a proxy for disease. The method of normalizing erythrocyte aggregation described herein may therefore have a marked improvement on the clinical outcome.

Some aspects relate to a machine implemented method to improve blood (or it's components) rheological parameters using low-intensity millimeter-wave radiation when the blood or its components (e.g. Red Blood Cells) are outside the organism. Some aspects relate to a machine implemented method to apply low-intensity millimeter waves to blood (or it's components) prior to transfusion. Some aspects relate to a machine implemented method to expose blood (or it's components) to electro-magnetic radiation at any frequency in range of 30 GHz to 300 GHz for a pre-defined duration.

Certain embodiments are described herein as numbered examples 1, 2, 3, etc. These numbered examples are provided as examples only and do not limit the subject technology.

Example 1 is an apparatus comprising: a container storing blood; means for attaching a device to the container; and means for emitting electromagnetic millimeter wave radiation on the container for a predetermined period of time.

In Example 2, the subject matter of Example 1 includes, wherein the blood comprises red blood cells or plasma.

In Example 3, the subject matter of Examples 1-2 includes, wherein the predetermined period of time is programmed by a user.

In Example 4, the subject matter of Examples 1-3 includes, wherein the container comprises a blood storage unit external to and distinct from a human body.

In Example 5, the subject matter of Examples 1-4 includes, wherein the means for attaching the device to the container comprises at least one magnet.

Example 6 is a system comprising: a container storing blood in an internal part of the container; and a device attached to the container at an external part of the container, the device emitting electromagnetic millimeter wave radiation on the container for a predetermined period of time.

In Example 7, the subject matter of Example 6 includes, wherein the blood comprises red blood cells or plasma.

In Example 8, the subject matter of Examples 6-7 includes, wherein the predetermined period of time is programmed by a user.

In Example 9, the subject matter of Examples 6-8 includes, wherein the container comprises a blood storage unit external to and distinct from a human body.

In Example 10, the subject matter of Examples 6-9 includes, wherein the device is attached to the container via at least one magnet.

Example 11 is a system comprising: a container storing packed red blood cells in an internal part of the container; a first magnet attached to the container at a first external part of the container; a second magnet attached to the container at a second external part of the container; and a millimeter wave emitter coupled to the second magnet, the millimeter wave emitter emitting electromagnetic millimeter wave radiation on the container for a predetermined period of time.

In Example 12, the subject matter of Example 11 includes, wherein the predetermined time period is programmed by a user.

In Example 13, the subject matter of Examples 11-12 includes, wherein the container comprises a blood storage unit external to and distinct from a human body.

Example 14 is an apparatus comprising means to implement of any of Examples 1-13.

Example 15 is a system to implement of any of Examples 1-13.

Example 16 is a method to implement of any of Examples 1-13.

What is claimed is:

1. A method of treatment of blood and its components comprising of electromagnetic radiation applied to blood when it is external to the body;

2. The method of claim 1, wherein the radiation duration is pre-determined;

3. The method of claim 1, wherein the radiation duration is dependent on sensor reading(s);

4. The method of claims 1 and 3, where sensor measures viscosity of blood;

5. The method of claims 1, 3, and 4, where sensor measures viscosity of blood by acoustic resonance;

6. The method of claims 1, 3, and 4, where sensor measures viscocity of blood by vibrational viscometery, rotational viscometery, capillary viscometery, or falling sphere viscometery;

7. The method of claims 1 and 3, where sensor measures viscocity of blood by Zahn cup;

8. The method of claims 1 and 3, where sensor measures opacity of blood;

9. The method of claim 1, 3 and 8, where sensor measures opacity of blood by optical measurements including standard “contrast-ratio method”;

10. The method of claim 1 and 3, where sensor measures color of blood;

11. The method of claims 1, 3, and 10, where sensor measures color by optical measurements. Color reading may be useful for determining blood conditions such as hemolysis, red cell contamination, lipemia, icterus, bacterial contamination, presence of particulate matter, and discoloration;

12. The method of claim 1, where parameters of electromagnetic radiation (frequency, intensity, pulse duration) are pre-determined and programmable by user;

13. The method of claim 1, where parameters of electromagnetic radiation (frequency, intensity, pulse duration) are pre-determined by the manufacturer;

14. The method of claim 1, where the parameters of electromagnetic radiation (frequency, intensity, pulse duration) are dependent on sensor readings (viscosity, opacity, color) via an algorithm;

15. The method of claim 1, where the pulse characteristics (duty cycle) vary based on battery life;

16. The method of claim 1, where the pulse characteristics (duty cycle) vary based on sensor reading(s) of blood parameters;

17. The method of claim 1, where the pulse characteristics (duty cycle) vary based on battery life and sensor reading(s) of blood parameters;

18. The method of claim 1, where the blood is irradiated while it moves relative to emitter;

19. The method of claim 1, where the blood is irradiated while it is stationary relative to the emitter;

20. The method of claim 1, where the emitter moves relative to the blood (e.g. scanning or spiral projection);

21. The method of claim 1, where rays of electromagnetic (EM) radiation moves, whereas both blood and emitter are stationary with regard to each other;

22. The method of claim 1, where the electromagnetic (EM) radiation is conical shape, cylindrical, or oval shape;

23. The method of claim 1, where there are multiple electromagnetic (EM) radiation rays acting on the blood simultaneously;

24. The method of claim 1, where the electromagnetic rays are at different frequencies, or intensities, or duty cycles;

25. The method of claim 1, where the processed blood is returned to the organism immediately (e.g. dialysis);

26. The method of claim 1, where the processed blood is stored and latter infused into the body;

27. The method of claim 1, where sensor readings are taken before and after electromagnetic (EM) radiation of blood to access blood changes with regard to viscosity, opacity, and/or color;

28. The method of claims 1 and 25, where concentration of heparin or other chemical or medicinal anti-coagulation products are infused into the blood dependent on blood viscosity reading(s) post electromagnetic (EM) radiation;

29. The method of claim 1 and 25, where automatic system shut-down is triggered due to sensor (viscosity, opacity, color) reading(s) and emergency room or other third party is automatically contacted for medical assistance;

30. The method of claims 1 and 26, where the processed blood is returned to the donor;

31. The method of claims 1 and 26, where the processed blood is infused to an individual who is not the donor (e.g. blood transfusion);

32. An apparatus comprising: a chamber that may contain blood or its components, and is at least partially made out of dielectric material; and emitter of electromagnetic millimeter or sub-millimeter wave radiation that is external to said chamber;

33. The apparatus of claim 32, wherein in one embodiment the chamber consists of catheter, and the emitters are placed on the catheter;

34. The apparatus of claim 32, wherein the device is attached to the catheter using at least one magnet;

35. The apparatus of claim 32, wherein an apparatus compromising: a catheter; emitter(s) attached to the catheter;

36. The apparatus of claim 32, wherein the emitting surface is coiled around the catheter;

37. The apparatus of claim 32, wherein the emitter(s) operate for a predetermined period of time that is programmed by a user or set by the manufacturer;

38. The apparatus of claim 32, wherein the emitter(s) operate for a period of time dependent on sensor reading(s) that may include viscosity, opacity, and/or color;

39. The apparatus of claim 32, wherein the emitter(s) parameters (frequency, intensity, pulse duration) may change based on sensor reading(s) including viscocity, opacity, and/or color in accordance with an algorithm. This may enable person-specific optimal settings to be achieved in terms of defining operation parameters most conducive to rapid normalization of blood parameters;

40. The apparatus of claim 32, wherein the emitting surface is covered by an insulating or reflective material to increase internal reflectivity of millimeter waves or sub-millimeter waves;

41. The apparatus of claim 32, wherein the emitter is inside the chamber;

42. The apparatus of claims 32 and 41, wherein there are multiple emitters that irradiate the moving blood with electromagnetic (EM) waves;

43. The apparatus of claims 32 and 41, where the emitters direct rays of electromagnetic (EM) waves in different directions.

44. The apparatus of claims 32 and 41, where the emitters direct rays of electromagnetic (EM) waves with different parameters (frequency, intensity, pulse duration);

45. The apparatus of claims 32 and 41, where the emitters are attached to a central rod and rotate such that irradiated volume changes dynamically;

46. The apparatus of claims 32 and 41, where the emitting surface is coiled so as to cover more surface area;

47. The apparatus of claim 32, wherein in one embodiment chamber consists of syringe, and the emitters are placed on the syringe;

48. The apparatus of claim 32, wherein in one embodiment chamber consists of container comprising a blood storage unit external to and distinct from the human body;

49. The apparatus of claims 32 and 48, wherein the container has magnetically attached electromagnetic (EM) emitter or emitters to it;

50. The apparatus of claims 32 and 48, wherein the container has a plate attached that distributes the blood to a thin (2-3 mm) plate that can easily be irradiated by electromagnetic (EM) radiation from emitter(s);

51. An apparatus compromising: a dialysis machine including an emitter that generates millimeter or sub-millimeter wave radiation in a continuous or a periodic fashion;

52. The apparatus of claim 51, wherein one embodiment the emitting surface is integrated into one of the components;

53. The apparatus of claims 51 and 52, wherein one embodiment the emitting surface is integrated into the filter element;

54. The apparatus of claims 51 and 52, wherein one embodiment the emitting surface is integrated into the catheter element;

55. The apparatus of claims 51 and 52, wherein one embodiment the emitting surface is integrated into the blood pump element;

56. The apparatus of claims 51 and 52, wherein one embodiment the emitting surface is integrated into any tubing element of the machine;

57. The apparatus of claims 51 and 52, wherein one embodiment the emitting surface is integrated into the heparin pump element;

58. The apparatus of claims 51 and 52, wherein one embodiment the emitting surface is integrated into any pressure monitors of the machine;

59. An apparatus compromising: sensor detectors to measure and monitor blood coagulation/viscosity before irradiation and after irradiation; these sensor detectors are attached to catheter; emitter of millimeter or sub-millimeter wave radiation that operates in a continuous or a periodic fashion; control of emitter parameters based on feedback from sensor detector(s);

60. The apparatus of claim 59 wherein in one embodiment apparatus compromising: acoustic resonance detectors to measure and monitor blood coagulation/viscosity before irradiation and after irradiation; resonance detectors attached to catheter; emitter of millimeter or sub-millimeter wave radiation that operates in a continuous or a periodic fashion; control of emitter parameters based on feedback from acoustic resonance detector(s);

61. The apparatus of claim 59 wherein in one embodiment apparatus compromising: optical detector(s) to measure and monitor blood coagulation/viscosity before irradiation and/or after irradiation; optical detector(s) connected to catheter; electromagnetic (EM) emitter that operates in a continuous or a periodic fashion; controller of emitter parameters based on feedback from optical detector(s);

62. An apparatus compromising: an article of clothing (pants, shirt, armband, leg-band, socks) to be worn with integrated millimeter or sub-millimeter wave emitter(s) to decrease edema and/or reduce pain, and/or reduce stiffness, and/or increase blood circulation;

63. An apparatus compromising: a body-suit to be worn with integrated millimeter or sub-millimeter wave emitter(s) to decrease edema and/or reduce pain, and/or reduce stiffness, and/or increase blood circulation to affected areas;

64. The apparatus of claim 63, wherein in one embodiment the control mechanism of effect localization is determined by stretch detectors;

65. The apparatus of claim 63, wherein one embodiment the control mechanism of effect localization is determined by optical detectors;

66. The apparatus of claim 63, wherein one embodiment the control mechanism of effect localization is determined by acoustic detectors.

67. An apparatus comprising: a stent; and an electromagnetic (EM) emitter integrated into the stent;

68. An apparatus compromising: a suction cup, cast, or other adherence to body via mechanical means; emitter of millimeter or sub-millimeter wave radiation in a continuous or a periodic fashion;

69. The apparatus of claim 68, compromising: a suction cup, cast, or other adherence to body via mechanical means; emitter of millimeter or sub-millimeter wave radiation in a continuous or a periodic fashion; and controller of emitter parameters (frequency, intensity, pulse duration, and possibly including duration);

70. An apparatus compromising: mesh of millimeter or sub-millimeter wave emitters that produce electromagnetic (EM) radiation in a continuous or a periodic fashion;

71. Apparatus of claim 70, compromising: mesh of millimeter or sub-millimeter wave emitters that produce electromagnetic (EM) radiation in a continuous or a periodic fashion; and controller of emitter parameters (frequency, intensity, pulse duration, duration).

72. An apparatus compromising: electrically powered massage device or chair with integrated mesh of millimeter or sub-millimeter wave emitters that produce electromagnetic radiation in a continuous or a periodic fashion; controller of emitter parameters. Massage device may be either Shiatsu (kneading massager with rolling balls) or vibration massager.

73. The apparatus of claim 72, wherein an apparatus compromising: infrared or heat massager; millimeter or sub-millimeter wave emitters that produce electromagnetic radiation in a continuous or a periodic fashion; and controller of emitter parameters (frequency, intensity, pulse duration, duration).
